(19) United States
(12) Patent Application Publication
(10) Pub. No.: US 2023/0125950 A1
(43) Pub. Date: Apr. 27, 2023

(54) METHOD AND DEVICE FOR SEPARATION AND PURIFICATION OF GLYCOLIC ACID BY RECTIFICATION-CRYSTALLIZATION COUPLING PROCESS AND USE

(71) Applicant: China University of Petroleum (East China), Qingdao City (CN)

(72) Inventors: Xin JIN, Qingdao City (CN); Yuangao WANG, Qingdao City (CN); Zhenchao YOU, Qingdao City (CN); Wenjuan YAN, Qingdao City (CN); Kexin MENG, Qingdao City (CN); Huiduan GE, Qingdao City (CN); Guangyu ZHANG, Qingdao City (CN); Youhai JIN, Qingdao City (CN); Chaohe YANG, Qingdao City (CN); Feng DU, Qingdao City (CN)

(21) Appl. No.: 17/967,251

(22) Filed: Oct. 17, 2022

(30) Foreign Application Priority Data
Oct. 21, 2021 (CN) 202111228539.9

(51) Int. Cl. C07C 51/43; B01D 3/14; B01D 3/10; B01D 9/00

(52) U.S. Cl. CPC C07C 51/43; B01D 3/143; B01D 3/10; B01D 9/0004; C07B 2200/13; B01D 2009/0086

(57) ABSTRACT

The present disclosure belongs to the technical field of separation and purification of glycolic acid, and in particular, to a method and device for separation and purification of glycolic acid by a rectification-crystallization coupling process and use. Bio-based platform compound molecules are used as raw materials to synthesize the glycolic acid, and the obtained crude glycolic acid is separated and purified using the rectification-crystallization coupling process to obtain high-purity glycolic acid. The method initiates system separation and purification under a new glycolic acid synthesis route, which has the difficulty that the glycolic acid is easy to polymerize during concentration, so there are technical barriers to equipment design of vacuum rectification and adjustment of process parameters. In addition, during crystallization, there are technical barriers to equipment design of a crystallization kettle and adjustment of process parameters.

### CROSS REFERENCE TO RELATED APPLICATION

This patent application claims priority of Chinese Patent Application No. 202111228539.9, filed on Oct. 21, 2021, the disclosure of which is incorporated by reference herein in its entirety as part of the present application.

### TECHNICAL FIELD

The present disclosure belongs to the technical field of separation and purification of glycolic acid, and in particular, to a method and device for separation and purification of glycolic acid by a rectification-crystallization coupling process and use.

### BACKGROUND

At present, glycolic acid, also known hydroxyacetic acid, is a colorless and easily deliquescent crystal. The glycolic acid is easily soluble in water and organic solvents such as methanol, ethanol, and ethyl acetate, slightly soluble in ether, and insoluble in hydrocarbons. The glycolic acid has the dual nature of alcohol and acid, and decomposes when heated to a boiling point.

At present, the glycolic acid is mainly prepared through coal, oil and natural gas routes, which require high temperature and high pressure operating conditions and significant energy consumption. With the proposal of carbon peak and carbon balanced programme, these technical routes with high CO₂ emissions are not in line with the current development trend. However, the route of synthesizing the glycolic acid based on bio-based platform compound molecules through a green and low-energy route has been developed, but impurities in the obtained glycolic acid are different from those of the traditional route.

At present, molecular rectification is mainly used in the glycolic acid separation technology, which has disadvantages such as complicated operation, high equipment investment, and low separation efficiency. The separation technology of the glycolic acid system synthesized by the bio-based route has not yet been proposed.

Through the above analysis, the problems existing in the prior art are summarized as follows:

(1) Since the glycolic acid is easy to polymerize during concentration, in the prior art, the equipment of vacuum rectification and process parameters cannot be effectively adjusted, resulting in poor separation and purification effect of the glycolic acid.

(2) During crystallization of the glycolic acid, the equipment of a crystallization kettle and the process parameters are not adjusted, resulting in poor separation and purification effect of the glycolic acid.

(3) In the prior art, the technology for preparing the glycolic acid is cumbersome, low in work efficiency, and cost-ineffective.

The difficulty of solving the above problems and defects is that a new bio-based glycolic acid separation and purification process needs to be proposed to simulate the procedure and optimize the process, which is mainly reflected in the design of the coupling device and the adjustment of process parameters. The significance of solving the above problems and defects is that the separation and purification effect of the bio-based glycolic acid can be improved, and process guidance is provided for the purification of other bio-based alkyd acids.

### SUMMARY

To overcome the problems in the related art, examples of the present disclosure provides a method and device for separation and purification of glycolic acid by a rectification-crystallization coupling process and use, and in particular, to a method for separation and purification of bio-based glycolic acid by a rectification-crystallization coupling process.

The technical solutions are as follows: a method for separation and purification of bio-based glycolic acid by a rectification-crystallization coupling process, including the following steps:

Step I, crude glycolic acid may be prepared using bio-based platform compound molecules as raw materials. Specifically, the platform compound molecules may be subjected to green transformation with a supported catalyst to synthesize the crude glycolic acid. For example, a reaction may be conducted at 80° C. for 10 h with a platinum on carbon (Pt/C) catalyst and using ethylene glycol bio-based platform compound molecules as the raw materials to obtain a crude glycolic acid solution.

Step II, the obtained crude glycolic acid may be subjected to rectification concentration to be greater than or equal to 70 wt. %. Specifically, the crude glycolic acid solution may be concentrated in a rectification column using vacuum rectification technology. For example, a crude glycolic acid solution with a mass fraction of 35 wt. % may be subjected to vacuum rectification. The rectification column may have a feed temperature of 30° C., a plate number of 25, a reflux ratio of 0.32, a bottom temperature of 60° C., and an absolute pressure of 0.29 MPa.

Step III, cooling crystallization and filtration may be conducted on the concentrated solution to obtain high-purity glycolic acid crystals. Specifically, the glycolic acid may be crystallized in a crystallization kettle coupled in series with the rectification column, and filtered and separated to obtain high-purity glycolic acid. For example, the obtained concentrated solution of the glycolic acid may be crystallized in the crystallization kettle at −15° C. under a cooling rate of 0.5° C./min and a stirring rate of 400 r/min, and 1 wt. % of glycolic acid seed crystals is added.

In one example, the bio-based platform compound molecules in step I may include one or a mixture of two or more selected from the group consisting of ethylene glycol, glyoxal, and oxalic acid diol aldehyde compounds, glycerol and butanediol polyol aldehyde compounds, and cellulose.

In one example, the crude glycolic acid in step I may include one or a mixture of two or more selected from the group consisting of glycolic acid, glycolaldehyde, ethylene glycol, glyoxal, glyoxylic acid, oxalic acid, formic acid, sorbitol, propylene glycol, butanediol, cellulose, glycerol, and lactic acid polyol aldehyde compounds.

In one example, the crude glycolic acid in step I may have a concentration of 0.5-99 wt. %.

In one example, methods for the rectification concentration in step II may include one or more of ordinary distillation, atmospheric rectification, vacuum rectification, and molecular rectification in series. The rectification concentration may be conducted at 20° C. to 250° C. under a rectification pressure (absolute pressure) of 0-10 MPa.

In one example, methods for crystallization in step III may include one or more of cooling crystallization, evaporative crystallization, sublimation crystallization, and recrystallization in series.

In one example, the cooling crystallization and the recrystallization may be conducted at −20° C. to 40° C. under a cooling rate of 0.1-20° C./min and a stirring rate of 100-1,500 r/min during cooling, and a percentage of a mass of glycolic acid seed crystals added to that of the glycolic acid in the concentrated solution may be 0.01-20%.

The evaporative crystallization and the sublimation crystallization may be conducted at −20° C. to 200° C.

In one example, preferably, the present disclosure transforms the bio-based platform compound molecules into the glycolic acid in a mild and green manner. The obtained crude glycolic acid is concentrated by vacuum rectification at a vacuum temperature of 50° C. under an absolute pressure of 0.5 MPa during vacuum, and the crude glycolic acid is concentrated to 70 wt. %. Cooling crystallization is conducted on the obtained concentrated solution at −20° C. under a cooling rate of 0.5° C./min and a stirring rate of 800 r/min, and a percentage of a mass of glycolic acid seed crystals added to that of the glycolic acid in the concentrated solution is 2%.

Another objective of the present disclosure is to provide a glycolic acid, obtained by the method for separation and purification of bio-based glycolic acid by a rectification-crystallization coupling process.

Another objective of the present disclosure is to provide a device for separation and purification by rectification and crystallization coupling, including: a vacuum rectification column and a crystallization kettle.

A rectification column plate is welded inside the vacuum rectification column including an upper part connected to a condenser through a tubing and a lower part connected to a reboiler and the crystallization kettle through tubings. A crystallization kettle temperature controller and a condensed water circulator are arranged at an upper part of the crystallization kettle. A mechanical stirring device is arranged inside the crystallization kettle.

Another objective of the present disclosure is to provide use of the method for separation and purification of bio-based glycolic acid by a rectification-crystallization coupling process in preparation of rust removers and detergents.

By combining all the technical solutions above, the present disclosure has the following advantages and positive effects.

For the glycolic acid of the present disclosure, bio-based platform compound molecules are used as raw materials to synthesize the glycolic acid, and the obtained crude glycolic acid is separated and purified using the rectification-crystallization coupling process to obtain high-purity glycolic acid.

The method initiates system separation and purification under a new glycolic acid synthesis route, which has the difficulty that the glycolic acid is easy to polymerize during concentration, so there are technical barriers to equipment design of vacuum rectification and adjustment of process parameters. In addition, during crystallization, there are technical barriers to equipment design of a crystallization kettle and adjustment of process parameters. The raw materials for the purification and separation of bio-based glycolic acid come from a special synthesis route, and there are catalyst design barriers that are difficult to overcome. For the purification and separation section, the easy polymerization property of glycolic acid leads to technical barriers to the selection of separation and purification process parameters and the coupling mode of the device.

The method has the advantages of simple and convenient operation process, good separation and purification effect, and high working efficiency. The method is cost-effective and has a relatively high market prospect.

Experiments show that the glycolic acid obtained by the present disclosure has a purity about 91.5-99.1%, which is greatly improved compared with the prior art.

It should be understood that the above general description and the following detailed description are only exemplary and explanatory, and should not be construed as a limitation to the present disclosure.

### BRIEF DESCRIPTION OF THE DRAWINGS

The accompanying drawings incorporated into the specification and constituting part of the specification illustrate the examples of the present disclosure, and serve, together with the specification, to explain the principles of the present disclosure.

FIG. 1 is a flow chart of a method for separation and purification of bio-based glycolic acid by a rectification-crystallization coupling process provided by an example of the present disclosure; and

FIG. 2 is a schematic structural diagram of a device for separation and purification by rectification and crystallization coupling provided by an example of the present disclosure.

Reference numerals: A, vacuum rectification column; B, crystallization kettle; 1, rectification column plate; 2, condenser; 3, reboiler; 4, crystallization kettle temperature controller; 5, condensed water circulator; and 6, mechanical stirring device.

### DETAILED DESCRIPTION OF THE EMBODIMENTS

To enable the objectives, features, and advantages mentioned above of the present disclosure to be more apparent and easily understood, specific implementations of the present disclosure will be described in detail below in conjunction with the drawings. The following describes many details in order to provide a thorough understanding of the present disclosure. However, the present disclosure can be implemented in many other ways other than those described herein, and those skilled in the art can make similar improvements without departing from the connotation of the present disclosure, and thus the present disclosure is not limited to the specific implementations disclosed below.

All technical and scientific terms used in the present disclosure have the same meaning as commonly understood by those skilled in the technical field of the present disclosure. The terms used in the specification of the present disclosure are only for the purpose of describing specific examples, rather than to limit the present disclosure. The term “and/or” used herein includes any and all combinations of one or more of the associated listed items.

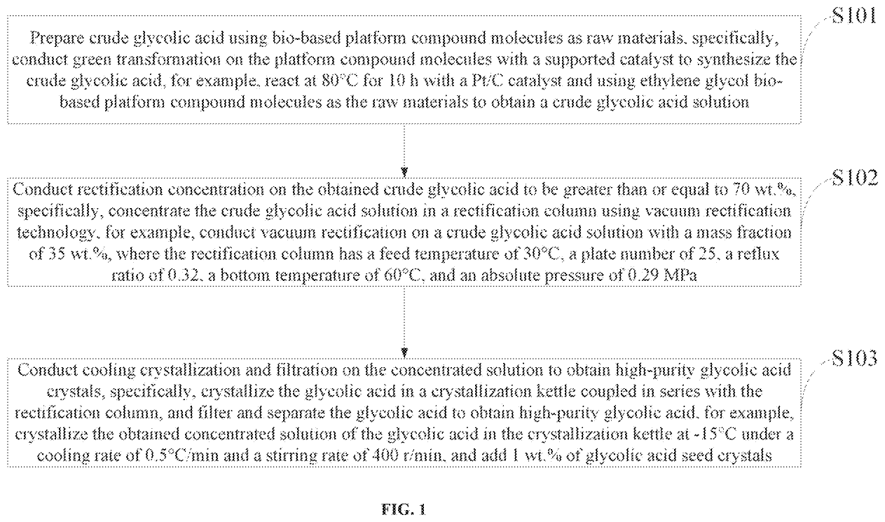

As shown in FIG. 1, the present disclosure provides a method for separation and purification of bio-based glycolic acid by a rectification-crystallization coupling process, including the following steps.

S101, crude glycolic acid is prepared using bio-based platform compound molecules as raw materials. Specifically, the platform compound molecules are subjected to green transformation with a supported catalyst to synthesize the crude glycolic acid. For example, a reaction is conducted at 80° C. for 10 h with a Pt/C catalyst and using ethylene glycol bio-based platform compound molecules as the raw materials to obtain a crude glycolic acid solution.

S102, the obtained crude glycolic acid is subjected to rectification concentration to be greater than or equal to 70 wt. %. Specifically, the crude glycolic acid solution is concentrated in a rectification column using vacuum rectification technology. For example, a crude glycolic acid solution with a mass fraction of 35 wt. % is subjected to vacuum rectification. The rectification column has a feed temperature of 30° C., a plate number of 25, a reflux ratio of 0.32, a bottom temperature of 60° C., and an absolute pressure of 0.29 MPa.

S103, cooling crystallization and filtration are conducted on the concentrated solution to obtain high-purity glycolic acid crystals. Specifically, the glycolic acid is crystallized in a crystallization kettle B coupled in series with the rectification column, and filtered and separated to obtain high-purity glycolic acid. For example, the obtained concentrated solution of the glycolic acid is crystallized in the crystallization kettle B at −15° C. under a cooling rate of 0.5° C./min and a stirring rate of 400 r/min, and 1 wt. % of glycolic acid seed crystals is added.

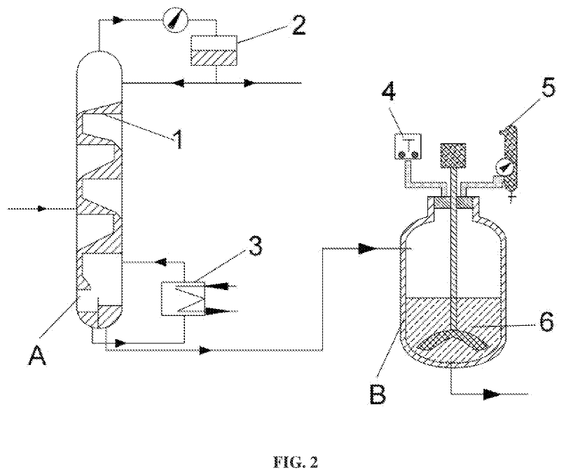

In an example of the present disclosure, as shown in FIG. 2, a device for separation and purification by rectification and crystallization coupling is further provided, composed of a vacuum rectification column A and a crystallization kettle B, and specifically including: a rectification column plate 1, a condenser 2, a reboiler 3, a crystallization kettle temperature controller 4, a condensed water circulator 5, and a mechanical stirring device 6. The crude glycolic acid is put into the rectification column plate 1. The glycolic acid crystals are taken out from the bottom of the crystallization kettle B. The rectification column plate 1 is welded inside the vacuum rectification column A including an upper part connected to the condenser 2 through a tubing and a lower part connected to the reboiler 3 and the crystallization kettle B through tubings. The crystallization kettle temperature controller 4 and the condensed water circulator 5 are arranged at an upper part of the crystallization kettle B. The mechanical stirring device 6 is arranged inside the crystallization kettle B.

The technical solutions of the present disclosure will be further described below in conjunction with specific examples.

### Example 1

A method for separation and purification of bio-based glycolic acid by a rectification-crystallization coupling process included the following steps.

Cellulose was subjected to one-step hydrothermal conversion with a tungsten/nickel catalyst at 200° C. under an initial hydrogen pressure of 6 MPa to obtain ethylene glycol, and the ethylene glycol was subjected to one-step hydrothermal conversion with a Pt catalyst supported on activated carbon (Pt/AC) at 80° C. under an initial oxygen pressure of 2 MPa to obtain crude glycolic acid.

The obtained crude glycolic acid was concentrated by vacuum rectification at a vacuum temperature of 50° C. under an absolute pressure of 0.5 MPa during vacuum, and the crude glycolic acid was concentrated to 70 wt. %.

Cooling crystallization was conducted on the obtained concentrated solution at −20° C. under a cooling rate of 0.5° C./min and a stirring rate of 800 r/min, and a percentage of a mass of glycolic acid seed crystals added to that of the glycolic acid in the concentrated solution was 2%.

Thus, the high-purity glycolic acid crystals were obtained.

### Examples 2 to 10

According to the method for separation and purification of glycolic acid in Example 1, the temperature and pressure of the vacuum rectification used were adjusted, and the other parameters were the same. The glycolic acid was separated and purified. The yield and purity of the glycolic acid were recorded, as shown in Table 1.

TABLE 1

| | T, ° C. | P, MPa | Yield, % | Purity, % |
|---|---|---|---|---|
| Example 1 | 40 | 0.05 | 56.7 | 91.5 |
| Example 2 | 40 | 0.1 | 70.5 | 93.9 |
| Example 3 | 40 | 0.2 | 81.4 | 92.7 |
| Example 4 | 40 | 0.5 | 60.6 | 95.8 |
| Example 5 | 40 | 1.0 | 66.9 | 96.8 |
| Example 6 | 30 | 0.1 | 59.9 | 93.4 |
| Example 7 | 50 | 0.1 | 72.3 | 95.8 |
| Example 8 | 60 | 0.1 | 76.9 | 96.5 |
| Example 9 | 70 | 0.1 | 78.5 | 98.7 |
| Example 10 | 80 | 0.1 | 89.4 | 99.1 |

### Examples 11 to 20

According to the method for separation and purification of glycolic acid in Example 1, the cooling rate, crystallization temperature, and rotational speed of the cooling crystallization used were adjusted, and the other parameters were the same. The glycolic acid was separated and purified. The yield and purity of the glycolic acid were recorded, as shown in Table 2.

TABLE 2

| | t, ° C./min | T, ° C. | R, r/min | Yield, % | Purity, % |
|---|---|---|---|---|---|
| Example 11 | 0.5 | −5 | 500 | 56.8 | 93.5 |
| Example 12 | 0.5 | −5 | 500 | 69.4 | 96.7 |
| Example 13 | 0.5 | −5 | 500 | 68.9 | 94.9 |
| Example 14 | 1 | −5 | 500 | 75.9 | 95.6 |
| Example 15 | 1 | −5 | 500 | 78.4 | 97.2 |
| Example 16 | 1 | −20 | 800 | 84.2 | 98.3 |
| Example 17 | 5 | −20 | 800 | 85.5 | 96.1 |
| Example 18 | 5 | −20 | 800 | 87.3 | 93.6 |
| Example 19 | 5 | −20 | 800 | 89.1 | 94.7 |
| Example 20 | 10 | −20 | 800 | 88.4 | 95.2 |

### Example 21

A method for separation and purification of bio-based glycolic acid by a rectification-crystallization coupling process included the following steps.

Glycerol was subjected to one-step hydrothermal conversion with a Pt—Fe/CeO₂ catalyst at 100° C. under an initial hydrogen pressure of 1 MPa to obtain crude glycolic acid.

The obtained crude glycolic acid was concentrated by vacuum rectification at a vacuum temperature of 50° C. under an absolute pressure of 0.5 MPa during vacuum, and the crude glycolic acid was concentrated to 70 wt. %.

Cooling crystallization was conducted on the obtained concentrated solution at −20° C. under a cooling rate of 0.5° C./min and a stirring rate of 800 r/min, and a percentage of a mass of glycolic acid seed crystals added to that of the glycolic acid in the concentrated solution was 2%.

Thus, the high-purity glycolic acid crystals were obtained.

### Example 22

A method for separation and purification of bio-based glycolic acid by a rectification-crystallization coupling process included the following steps. Methyl glycolate was hydrolyzed to obtain crude glycolic acid in one step.

The obtained crude glycolic acid was concentrated by vacuum rectification at a vacuum temperature of 50° C. under an absolute pressure of 0.5 MPa during vacuum, and the crude glycolic acid was concentrated to 70 wt. %.

Cooling crystallization was conducted on the obtained concentrated solution at −20° C. under a cooling rate of 0.5° C./min and a stirring rate of 800 r/min, and a percentage of a mass of glycolic acid seed crystals added to that of the glycolic acid in the concentrated solution was 2%.

Thus, the high-purity glycolic acid crystals were obtained.

### Example 23

A method for separation and purification of bio-based glycolic acid by a rectification-crystallization coupling process included the following steps.

Ethylene glycol was subjected to one-step hydrothermal conversion with an Au/NaY catalyst at 95° C. under an initial hydrogen pressure of 1 MPa to obtain crude glycolic acid.

The obtained crude glycolic acid was concentrated by vacuum rectification at a vacuum temperature of 50° C. under an absolute pressure of 0.5 MPa during vacuum, and the crude glycolic acid was concentrated to 70 wt. %.

Cooling crystallization was conducted on the obtained concentrated solution at −20° C. under a cooling rate of 0.5° C./min and a stirring rate of 800 r/min, and a percentage of a mass of glycolic acid seed crystals added to that of the glycolic acid in the concentrated solution was 2%.

Thus, the high-purity glycolic acid crystals were obtained.

The vacuum rectification temperature and pressure were adjusted according to the glycolic acid of different synthetic sources of Examples 24 to 31, and the other parameters were the same. The glycolic acid was separated and purified. The yield and purity of the glycolic acid were recorded, as shown in Table 3.

TABLE 3

| | Raw | | | | |
|---|---|---|---|---|---|
| | materials | | | | |
| | for | | | | |
| | synthesizing | | | | |
| | glycolic acid | T, ° C. | P, MPa | Yield, % | Purity, % |
| Example 24 | Cellulose | 50 | 0.1 | 58.3 | 92.3 |
| Example 25 | Glycerol | 50 | 0.1 | 56.8 | 96.1 |
| Example 26 | Methyl | 50 | 0.1 | 59.4 | 97.2 |
| | glycolate | | | | |
| Example 27 | Ethylene | 50 | 0.1 | 56.1 | 95.3 |
| | glycol | | | | |
| Example 28 | Cellulose | 70 | 0.05 | 70.4 | 97.1 |
| Example 29 | Glycerol | 70 | 0.05 | 75.3 | 98.5 |
| Example 30 | Methyl | 70 | 0.05 | 76.8 | 99.1 |
| | glycolate | | | | |
| Example 31 | Ethylene | 70 | 0.05 | 79.1 | 94.5 |
| | glycol | | | | |

The vacuum rectification temperature and pressure were adjusted according to the glycolic acid of different synthetic sources of Examples 32 to 39, and the other parameters were the same. The glycolic acid was separated and purified. The yield and purity of the glycolic acid were recorded, as shown in Table 4.

TABLE 4

| | Raw | | | | | |
|---|---|---|---|---|---|---|
| | materials | | | | | |
| | for | | | | | |
| | synthesizing | t, | T, | R, | Yield, | Purity, |
| | glycolic acid | ° C./min | ° C. | r/min | % | % |
| Example 32 | Cellulose | 1 | −5 | 200 | 61.4 | 93.5 |
| Example 33 | Glycerol | 1 | −5 | 200 | 68.1 | 96.1 |
| Example 34 | Methyl | 1 | −5 | 200 | 64.9 | 92.7 |
| | glycolate | | | | | |
| Example 35 | Ethylene | 1 | −5 | 200 | 69.6 | 98.2 |
| | glycol | | | | | |
| Example 36 | Cellulose | 5 | −15 | 600 | 81.4 | 94.1 |
| Example 37 | Glycerol | 5 | −15 | 600 | 85.9 | 95.7 |
| Example 38 | Methyl | 5 | −15 | 600 | 84.8 | 93.1 |
| | glycolate | | | | | |
| Example 39 | Ethylene | 5 | −15 | 600 | 89.2 | 94.9 |
| | glycol | | | | | |

Those skilled in the art can easily think of other implementation solutions of the present disclosure after considering the specification and practicing the disclosure herein. This application is intended to cover any variations, purposes or adaptive changes of the present disclosure. Such variations, purposes or applicable changes follow the general principle of the present disclosure and include common knowledge or conventional technical means in the technical field which is not disclosed in the present disclosure. The specification and examples are merely considered as illustrative, and the real scope and spirit of the present disclosure are pointed out by the claims.

It should be noted that the present disclosure is not limited to the precise structures described above and shown in the accompanying drawings, and can be modified and changed in many ways without departing from the scope of the present disclosure. The scope of the present disclosure is defined by the appended claims.

What is claimed is:

1. A method for separation and purification of bio-based glycolic acid by a rectification-crystallization coupling process, the method comprising:
synthesizing glycolic acid using bio-based platform compound molecules as raw materials; and
separating and purifying the obtained crude glycolic acid using the rectification-crystallization coupling process to obtain high-purity glycolic acid.

2. The method according to claim 1, further comprising the following steps:
step I, preparing the crude glycolic acid using the bio-based platform compound molecules as the raw materials: transforming the platform compound molecules with a supported catalyst to synthesize the crude glycolic acid;
step II, conducting rectification concentration on the obtained crude glycolic acid: concentrating a crude glycolic acid solution to be greater than or equal to 70 wt. % in a rectification column using vacuum rectification technology; and
step III, conducting cooling crystallization and filtration on the concentrated solution to obtain high-purity glycolic acid crystals: crystallizing the glycolic acid in a crystallization kettle coupled in series with the rectification column, and filtering and separating the crystallized glycolic acid to obtain the high-purity glycolic acid.

3. The method according to claim 2, wherein preparing the crude glycolic acid using the bio-based platform compound molecules as the raw materials in step I further comprises: reacting at 80° C. for 10 h with a platinum on carbon (Pt/C) catalyst and using ethylene glycol bio-based platform compound molecules as the raw materials to obtain the crude glycolic acid solution.

4. The method according to claim 1, wherein the bio-based platform compound molecules in step I comprise one or a mixture of two or more selected from the group consisting of ethylene glycol, glyoxal, and oxalic acid diol aldehyde compounds, glycerol and butanediol polyol aldehyde compounds, and cellulose.

5. The method according to claim 1, wherein the crude glycolic acid in step I comprises one or a mixture of two or more selected from the group consisting of glycolic acid, glycolaldehyde, ethylene glycol, glyoxal, glyoxylic acid, oxalic acid, formic acid, sorbitol, propylene glycol, butanediol, cellulose, glycerol, and lactic acid polyol aldehyde compounds.

6. The method according to claim 1, wherein conducting rectification concentration on the obtained crude glycolic acid in step II further comprises:
concentrating a crude glycolic acid solution with a mass fraction of 35 wt. % to be greater than or equal to 70 wt. % by vacuum rectification at 20° C. to 250° C. under 0-10 MPa; and the rectification column has a feed temperature of 30° C., a plate number of 25, a reflux ratio of 0.32, a bottom temperature of 60° C., and an absolute pressure of 0.29 MPa; and
methods for the rectification concentration comprise one or more of ordinary distillation, atmospheric rectification, vacuum rectification, and molecular rectification in series.

7. The method according to claim 1, wherein methods for crystallization in step III comprise one or more of cooling crystallization, evaporative crystallization, sublimation crystallization, and recrystallization in series; and
a process of conducting cooling crystallization and filtration on the concentrated solution to obtain high-purity glycolic acid crystals further comprises: crystallizing the obtained concentrated solution of the glycolic acid in the crystallization kettle, the cooling crystallization and the recrystallization are conducted at −20° C. to 40° C. under a cooling rate of 0.1-20° C./min and a stirring rate of 100-1,500 r/min during cooling, a percentage of a mass of glycolic acid seed crystals added to that of the glycolic acid in the concentrated solution is 0.01-20%; and the evaporative crystallization and the sublimation crystallization are conducted at −20° C. to 200° C.

8. A glycolic acid, obtained by the method according to claim 1.

9. The glycolic acid according to claim 8, wherein the method further comprises the following steps:
step I, preparing the crude glycolic acid using the bio-based platform compound molecules as the raw materials: transforming the platform compound molecules with a supported catalyst to synthesize the crude glycolic acid;
step II, conducting rectification concentration on the obtained crude glycolic acid: concentrating a crude glycolic acid solution to be greater than or equal to 70 wt. % in a rectification column using vacuum rectification technology; and
step III, conducting cooling crystallization and filtration on the concentrated solution to obtain high-purity glycolic acid crystals: crystallizing the glycolic acid in a crystallization kettle coupled in series with the rectification column, and filtering and separating the crystallized glycolic acid to obtain the high-purity glycolic acid.

10. The glycolic acid according to claim 8, wherein preparing the crude glycolic acid using the bio-based platform compound molecules as the raw materials in step I further comprises: reacting at 80° C. for 10 h with a platinum on carbon (Pt/C) catalyst and using ethylene glycol bio-based platform compound molecules as the raw materials to obtain the crude glycolic acid solution.

11. The glycolic acid according to claim 8, wherein the bio-based platform compound molecules in step I comprise one or a mixture of two or more selected from the group consisting of ethylene glycol, glyoxal, and oxalic acid diol aldehyde compounds, glycerol and butanediol polyol aldehyde compounds, and cellulose.

12. The glycolic acid according to claim 8, wherein the crude glycolic acid in step I comprises one or a mixture of two or more selected from the group consisting of glycolic acid, glycolaldehyde, ethylene glycol, glyoxal, glyoxylic acid, oxalic acid, formic acid, sorbitol, propylene glycol, butanediol, cellulose, glycerol, and lactic acid polyol aldehyde compounds.

13. The glycolic acid according to claim 8, wherein conducting rectification concentration on the obtained crude glycolic acid in step II further comprises: concentrating a crude glycolic acid solution with a mass fraction of 35 wt. % to be greater than or equal to 70 wt. % by vacuum rectification at 20° C. to 250° C. under 0-10 MPa; and the rectification column has a feed temperature of 30° C., a plate number of 25, a reflux ratio of 0.32, a bottom temperature of 60° C., and an absolute pressure of 0.29 MPa; and
methods for the rectification concentration comprise one or more of ordinary distillation, atmospheric rectification, vacuum rectification, and molecular rectification in series.

14. The glycolic acid according to claim 8, wherein crystallization in step III comprise one or more of cooling crystallization, evaporative crystallization, sublimation crystallization, and recrystallization in series; and
a process of conducting cooling crystallization and filtration on the concentrated solution to obtain high-purity glycolic acid crystals further comprises: crystallizing the obtained concentrated solution of the glycolic acid in the crystallization kettle, the cooling crystallization and the recrystallization are conducted at −20° C. to 40° C. under a cooling rate of 0.1-20° C./min and a stirring rate of 100-1,500 r/min during cooling, a percentage of a mass of glycolic acid seed crystals added to that of the glycolic acid in the concentrated solution is 0.01-20%; and the evaporative crystallization and the sublimation crystallization are conducted at −20° C. to 200° C.

15. A device for separation and purification by rectification and crystallization coupling implementing the method for separation and purification of bio-based glycolic acid by a rectification-crystallization coupling process according to claim 1, the device comprising:
a vacuum rectification column and a crystallization kettle;
a rectification column plate is welded inside the vacuum rectification column comprising an upper part connected to a condenser through a tubing and a lower part connected to a reboiler and the crystallization kettle through tubings; and
a crystallization kettle temperature controller and a condensed water circulator are arranged at an upper part of the crystallization kettle; and a mechanical stirring device is arranged inside the crystallization kettle.

16. The device according to claim 15, wherein the method further comprises the following steps:
step I, preparing the crude glycolic acid using the bio-based platform compound molecules as the raw materials: transforming the platform compound molecules with a supported catalyst to synthesize the crude glycolic acid;
step II, conducting rectification concentration on the obtained crude glycolic acid: concentrating a crude glycolic acid solution to be greater than or equal to 70 wt. % in a rectification column using vacuum rectification technology; and
step III, conducting cooling crystallization and filtration on the concentrated solution to obtain high-purity glycolic acid crystals: crystallizing the glycolic acid in a crystallization kettle coupled in series with the rectification column, and filtering and separating the crystallized glycolic acid to obtain the high-purity glycolic acid.

17. The device according to claim 15, wherein a process of preparing the crude glycolic acid using the bio-based platform compound molecules as the raw materials in step I further comprises: reacting at 80° C. for 10 h with a platinum on carbon (Pt/C) catalyst and using ethylene glycol bio-based platform compound molecules as the raw materials to obtain the crude glycolic acid solution.

18. The device according to claim 15, wherein the bio-based platform compound molecules in step I comprise one or a mixture of two or more selected from the group consisting of ethylene glycol, glyoxal, and oxalic acid diol aldehyde compounds, glycerol and butanediol polyol aldehyde compounds, and cellulose.

19. The device according to claim 15, wherein the crude glycolic acid in step I comprises one or a mixture of two or more selected from the group consisting of glycolic acid, glycolaldehyde, ethylene glycol, glyoxal, glyoxylic acid, oxalic acid, formic acid, sorbitol, propylene glycol, butanediol, cellulose, glycerol, and lactic acid polyol aldehyde compounds.

20. The device according to claim 15, wherein conducting rectification concentration on the obtained crude glycolic acid in step II further comprises: concentrating a crude glycolic acid solution with a mass fraction of 35 wt. % to be greater than or equal to 70 wt. % by vacuum rectification at 20° C. to 250° C. under 0-10 MPa; and the rectification column has a feed temperature of 30° C., a plate number of 25, a reflux ratio of 0.32, a bottom temperature of 60° C., and an absolute pressure of 0.29 MPa; and
methods for the rectification concentration comprise one or more of ordinary distillation, atmospheric rectification, vacuum rectification, and molecular rectification in series.
